(19) United States
(12) Patent Application Publication
(10) Pub. No.: US 2016/0137624 A1
(43) Pub. Date: May 19, 2016

(54) PROCESS FOR PREPARING PYRIDYLPYRAZOLE COMPOUNDS AND DERIVATIVES THEREOF FROM PYRIDYLHYDRAZINE

(71) Applicant: BASF SE, Ludwigshafen (DE)

(72) Inventors: Thomas ZIERKE, Hassloch (DE); Monika DOMRES, Frankenthal (DE); Christopher KORADIN, Grommersheim (DE); Karsten KOERBER, Eppelheim (DE)

(21) Appl. No.: 14/897,953

(22) PCT Filed: Jun. 17, 2014
(86) PCT No.: PCT/EP2014/062709

§ 371 Date: Dec. 11, 2015

(30) Foreign Application Priority Data
Jun. 20, 2013 (EP) 13173044.2

(51) Int. Cl. C07D 401/04

(52) U.S. Cl. CPC C07D 401/04

(57) ABSTRACT

The present invention relates to a process for preparing pyridylpyrazole compounds of the formula (I)

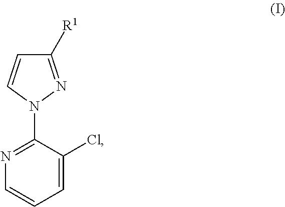

starting from pyridylhydrazine of formula (II)

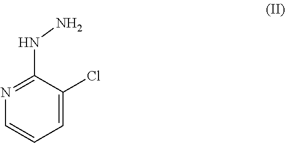

The present invention relates also to processes comprising further preceding and/or subsequent reaction steps, leading to anthranilamide pesticides or to precursors for them.

The present invention relates to a process for preparing pyridylpyrazole compounds and derivatives thereof, in particular pyridylpyrazole carbonyl compounds. It also relates to the use of these pyridylpyrazole carbonyl compounds for preparing anthranilamide derivatives that are useful pesticides. Therefore, pyridylpyrazole compounds are important precursors for anthranilamide derivates. Such compounds find use as pesticides, especially as insecticides, which are disclosed, for example, in WO 01/70671, WO 03/015518, WO 03/015519,

WO 03/016284, WO 03/016300, WO 03/024222, WO2003/062221, WO2003/027099, WO2004/067528, WO2003/106427, WO 06/000336; WO 06/068669, WO 07/043677, WO2008/126933, WO2008/126858, and WO2008/130021, and in WO2007/006670, WO2013/024009, WO2013/024010, WO2013/024003, WO2013/024004, WO2013/024005, WO2013/024006, WO2013/024169, WO2013/024170, WO2013/024171. WO2010/037688 describes a process for preparing NH-pyrazole compounds, e.g. starting from a vinyl ether and hydrazine. However, the subsequent reaction to pyridylpyrazole compounds suffer from several disadvantages, e.g. the dichloropyridine to be employed is expensive and must be employed in excess, the polar solvents to be used are expensive and hard to recover, and the reaction sequence tends to undesired side reactions.

It is an object of the present invention to provide alternative or improved processes for preparing pyridylpyrazole compounds and for preparing pyrazolecarboxamides or anthranilamides derived therefrom. These processes should be simple to carry out, require 4 or 3 or less steps and be suitable for the industrial scale production. The processes should have good yields and good product purity, and start from readily available starting materials. They should additionally be inexpensive and safe and be based on selective reactions. The object is achieved by the processes described in detail hereinafter.

In a first aspect, the present invention relates to a process for preparing a pyridylpyrazole compound of the formula (I)

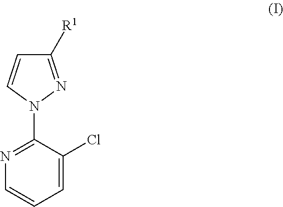

in which R¹ is selected from CF₃ and CHF₂;
comprising the step of reacting a compound of the formula (II)

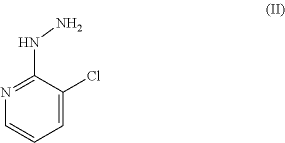

with a compound of formula (III)

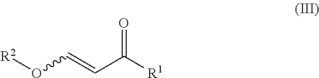

wherein R¹ is as defined above;
and R² is selected from C₁-C₆-alkyl, C₂-C₆-cycloalkyl, aralkyl and aryl;
in the presence of an acid.

This process is hereinafter also referred to as step (ii).

For the anthranilamide pesticides as mentioned above, a precursor is needed, which is a pyridylpyrazole compound of the type of the compound of formula (I). Usually, the preparation of this compound was achieved in literature by coupling of a 3-substituted NH-pyrazole with 2,3-dichloropyridine in the presence of potassium carbonate in DMF at 125° C. (Bioorg. Med. Chem. Lett. (2005) 4898-4906).

This method has some disadvantages, in view of a profitable industrial application: The process requires the absence of water; DMF cannot be recovered easily after work-up in water; 2,3-dichloropyridine has to be employed in excess in order to favour the selectivity. Nevertheless, the formation of 2-fold substituted pyridines as side products cannot be avoided, and the yield can hardly be improved.

Also WO2013/024008 and WO2013/076092 use the approach to synthesize the 3-substituted NH-pyrazole from e.g. ETFBO and hydrazine, followed by a coupling of the 3-substituted NH-pyrazole with 2,3-dichloropyridine. Based on 2,3-dichloropyridine as starting material in step 2, the overall yield is 57% (step1: 77.5%, step2: 74%). Based on the NH-pyrazole as starting material, the overall yield of compound I is 63% (step1: 77.5%; step2: 81.5%). Even if in step 1, the yield was assumed to be as high as 92% (as described in WO2010/037688, Solvay), the best overall yield, one can calculate (including step2: 81.5%), would be only 75%.

Processes on an industrial scale usually require higher yields, resulting also often in less purification problems.

A higher yield would be more economic and is therefore highly desirable.

An object of the present invention was therefore to provide an economical process for the preparation of the pyridylpyrazole compounds of the type of the compound of formula (I).

This object was achieved by the present new process route. This route takes advantage from reversing the steps, optionally including certain adaptation of the steps. The present invention relates to a process, wherein pyridylhydrazine II (obtainable e.g. from 2,3-dichloropyridine and hydrazine) is coupled with vinyl ethers of formula III.

The reactant compound of formula (II) can be obtained by procedures as known in the literature.

For example, it is known that dichloropyridine and hydrazine can be reacted to a compound of formula (II) in excellent yields, especially in yields over 90%, see e.g. JOC 35 S.810 (1970) for a reaction of 2,3-dichloropyridine with hydrazine hydrate. For example, EP441718 and CN102584694 describe yields between 92 and 98% for compound II. In the present application, see e.g. Example 2, the yield for step (ii) is 93.7%. The overall yield, starting from 2,3-dichloropyridine, is therefore 86-92%.

This is significantly higher than the maximum yield of 63 or 75% described in or calculated from literature.

As described above, WO2010/037688 describes a process for preparing NH-pyrazole compounds, e.g. starting from a vinyl ether and hydrazine. It has to be noted that WO2010/037688 does not describe the synthesis of N-heteroaryl-substituted pyrazoles, nor N-pyridyl-substituted pyrazoles (Y can be nitrogen, or even NHR3 wherein R3 is an alkyl aryl or aralkyl residue). There is no example in WO2010/037688 for an alkyl- or aryl-substituted (nor heteroaryl-substituted) hydrazine as starting material.

According to the literature, the reaction of compounds of formula (III) like ETFBO with phenylsubstituted (no heteroaryl) hydrazines does not lead to phenylpyrazole products (e.g. J. Heterocycl. Chem. 30, 1156 (1993)). The publication of Eur. J. Med. Chem. 2003, 38, p. 157 ff discloses the reaction of a para-substituted phenylhydrazine hydrochloride with ETFBO by heating in ethanole, yielding a 3-CF3-substituted N-Phenylpyrazole. In the experimental part (6.1.1. on page 164), it is described that a 60:40 mixture of the desired product with the 5-CF3- substituted isomer is obtained. The selectivity is low. The desired compound can be isolated only in 40% yield after chromatograophy. The reaction was repeated with the reactants of the present invention, see comparison example Cl. The low selectivity and moderate yield could be verified. The process is therefore probably not suitable for industrial application. Therefore, a person skilled in the art would be led away to use this reaction for a selective process for 3-haloalkyl substituted aryl or hetaryl pyrazoles.

The approach as used in Eur. J. Med. Chem. 2003, 38, p. 157 ff, is further developed in Tetrahedron 67 (2011)5663 for the ClCF₂ analogue of ETFBO, reacted with phenylhydrazine and 4-NO₂-phenylhydrazine. The reaction was repeated also in this case with the reactants of the present invention, see comparison example C₂. The desired product, in mixture with the undesired isomer, could be detected, but the main product is a different compound. The process is therefore probably not suitable for industrial application. Therefore, a person skilled in the art would be led away to use this reaction for a selective process for 3-haloalkyl substituted aryl or hetaryl pyrazoles.

The reaction with alkyl-substituted hydrazines leads to isomeric mixtures. Alternatively, more complex routes have to be used to arrive at substituted phenylpyrazoles, as described e.g. in Tetrahedron Lett. 2012 (53), p. 5488; Eur. J. Org. Chem. 2004, 695_709; Org. Biomol. Chem., 2009, 7, 2155-2161.

No attempt has been described to react N-Heteroaryl-substituted hydrazines with ETFBO. It is therefore highly surprising that the process according to the invention leads to compounds of formula (I), especially with high selectivity and in high yields, especially in higher yields than the known process with a different order of reaction steps.

The processes of the invention are associated with a series of advantages as they overcome the aforementioned shortcomings of the prior art processes. The processes of the invention, especially step (ii), provide the pyridylpyrazole compound of formula (I) in high yields and in excellent regioselectivity. Undesired side reactions leading to unwanted by-products are minimized. This makes purification easier, which can be done e.g. by distillation (or distillation/crystallization later in the process steps). Sometimes, the product can be employed in the next reaction step without purification. This prevents losses during work-up or purification, and this also saves time, resources and/or energy. Further advantages of the processes of the present invention are that the processes can be run at moderate temperatures. The solvents can be recovered and be re-used. The reagents to be used are safe and inexpensive, which is favourable in view of costs and safety aspects. The reactants are cheap and readily available or can be easily manufactured. Due to these properties, the processes are therefore suitable for production on an industrial scale, which is a further advantage.

The acid employed in the reaction referred to as step (ii) is a protonic acid and may be selected from inorganic or organic acids. In one embodiment, the acid may be selected from concentrated HCl, concentrated sulfuric acid, concentrated phosphoric acid, benzene sulfonic acid and p-toluene sulfonic acid. In one embodiment, the acid is selected from hydrochloric acid HCl, sulfuric acid H₂SO₄ and phosphoric acid H₃PO₄, preferably hydrochloric acid HCl and sulfuric acid H₂SO₄. In another embodiment, the acid may be selected from concentrated HCl and concentrated sulfuric acid H₂SO_{4.} In another embodiment, the acid is gaseous HCl. In one embodiment, the acid is an aqueous acid. Aqueous acid means a mixture of the respective acid with water. In one embodiment, where the respective acid is HCl, the amount of water may be from 63 to 75% or from 63 to 70%.

In one embodiment, the acid is concentrated hydrochloric acid. Concentrated hydrochloric acid may be understood as a concentration up to the saturated solution, which means at 20° C. that one liter of saturated HCl aqueous solution contains 720 g HCl. In another embodiment, the acid is concentrated sulfuric acid. Concentrated sulfuric acid may contain up to 98% sulfuric acid.

The amount of acid can be varied in broad ranges. It may e.g. be varied from 0.05 to 10 equivalents [=” eq” , in relation to the compound (II)], or from 0.1 to 5 eq, or from 0.1 to 3 eq, or from 0.15 to 3 eq, or from 0.15 to 2 eq. For example, it may e.g. be 0.15 to 1 eq in the case of sulfuric acid and up to 2 equivalents in the case of concentrated hydrochloric acid. In one embodiment, the acid is employed in an under-stoichiometric ratio with regard to compound (II). “Under-stoichiometric” ratio means that the number of equivalents is smaller than 1, e.g. 0.05 eq, 0.1 eq, 0.15 eq, 0.2 eq, 0.25 eq, 0.3 eq, 0.35 eq, 0.5 eq, 0.6 eq, 0.7 eq, 0.75 eq, 0.8 eq, 0.9 eq. In one embodiment, the number of equivalents is smaller than 0.5.

In one embodiment, the reaction is carried out in a solvent. In one embodiment, the reaction is carried out in an organic solvent which is selected from toluene, ethylbenzene, o-xylene, m-xylene, p-xylene, chlorobenzene, hexane, cyclohexane, methylcyclohexane, or a mixture thereof.

In one embodiment, the reaction is carried out in a solvent which is an aromatic solvent. In one embodiment, the aromatic solvent is selected from from toluene, ethylbenzene, o-xylene, m-xylene, p-xylene, chlorobenzene, or a mixture thereof, preferably toluene. In one embodiment, the reaction is carried out in a non-aromatic organic solvent. In one embodiment, the non- aromatic organic solvent is selected from hexane, cyclohexane, methylcyclohexane or a mixture thereof.

The temperature at which the reaction is carried out (reaction temperature) may be varied in broad ranges, which the person skilled in the art knows, often depends from the reflux temperature of the solvent to be used. In one embodiment, the reaction is carried out at a temperature between 15 to 150° C., or 20 to 150° C., or 20 to 120° C., or 25 to 120° C., or 30 to 120° C., or 40 to 120° C., or 50 to 120° C., or 60 to 120° C., or 70 to 120° C.

The duration time of the reaction varies depending on the amount of acid and depending on the reaction termperature. The end of the reaction can be monitored by methods known to the person skilled in the art, e.g. thin layer chromatography, HPLC. In one embodiment, the reaction is carried out under heating to reflux for up to 20 hours.

The conversion of compound of formula (II), (3-Chloro-2-pyridyl)hydrazine, with a compound of formula (III), [compound of formula (III) in the case of R²=ethyl: “ ETFBO” (4-ethoxy-1,1,1-trifluoro-but-3-en-2-one)], has not been described so far.

The result according to the invention is surprising, in view of the fact that the same reaction without addition of acid leads to the predominant formation of a compound of formula (IV)

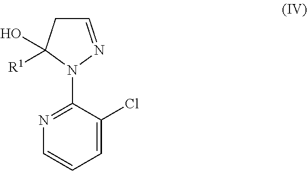

in which R¹ is selected from CF₃ and CHF₂.

In one embodiment, the invention relates to a compound of formula (IV),

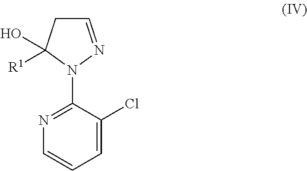

in which R¹ is selected from CF₃ and CHF₂.

By way of dehydratization (e.g. by temperature or acid addition), compounds of formula (IV) may be converted to compounds of formula (V):

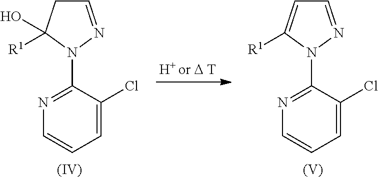

in which R¹ is selected from CF₃ and CHF₂.

The compound of formula (IV) is also formed under the conditions according to the invention, at the time of mixing of the reaction partners at room temperature (20 to 25° C.). Yet, upon reaction under heating to reflux in the presence of acid, the compound of formula (I) is obtained in high yields, The compound of formula (V) is formed only to a minor extent (side product).

The process according to the invention does not depend on the order of addition of the reaction partners. It is possible to provide the acid in the solvent, to which the compound of formula (II) is then added, or to provide the compound of formula (II) in the solvent, to which the acid is then added, after which the compound of formula (III) is added, e.g. at room temperature (20-25° C.). The compound of formula (III) can be added as one portion or in doses over time (continuous or a number of doses). It is also possible to add the compound of formula (III) only after heating of the provided reaction mixture. The compound of formula (III) can be added as a pure compound or as a solution in a solvent, preferably a solution in the selected solvent.

In a further embodiment, the order of addition is that the compounds of formula (II) and (III) are provided at 20-30° C. in the solvent, and subsequently the acid is added at 25-30° C. In a further embodiment, the order of addition is that the acid is provided in the solvent, and subsequently the compounds of formula (III) and (II) are added at room temperature (usually 20-25° C.).

In a further embodiment, the order of addition is that the acid is provided in the solvent, and subsequently the compound of formula (III) is added at room temperature (usually 20-25° C.), and subsequently the compound of formula (II) is added as the last component.

The person skilled in the art knows the best work-up of the reaction mixture after the end of the reaction. After cooling, the phase of reaction water, which usually contains the acid, is removed. The organic phase is washed with water, possibly under use of bases such as NaHCO₃, Na₂CO₃ oder NaOH, to achieve neutralization. Upon removal of the solvent (distillation, e.g. at low temperatures, under reduced pressure, possibly azeotropic removal of water), the compound of formula (I) is obtained in high yield as crude product. The compound of formula (I) may be employed as crude product in the next reaction step towards the insecticidal compounds described in the beginning. Alternatively, the compound of formula (I) may be purified by methods known to the person skilled in the art and may be employed as a pure compound in the next reaction step towards the insecticidal compounds described in the beginning.

In a preferred embodiment, the order of addition is: 1.) the compound of formula (II), 2.) the acid, e.g. sulfuric acid H₂SO₄, 3.) the compound of formula (III) at room temperature (usually 20 to 25° C.), 4.) heating to reflux.

In an alternative embodiment, the order of addition is: 1.) the compound of formula (II), and the acid, e.g. sulfuric acid H₂SO₄, 2.) heating to reflux for 1 to 2 hours, 3.) after heating according to 2, addition of the compound of formula (III).

In another alternative embodiment, the order of addition is: 1.) the compound of formula (III) and the acid, e.g. hydrochloric acid, 2.) the compound of formula (II) at room temperature (usually 20 to 25° C.), 3.) heating to reflux.

In another alternative embodiment, the order of addition is: 1.) the compound of formula (II) and the compound of formula (III) at room temperature (usually 20 to 25° C.), 2.) the acid, e.g. sulfuric acid H₂SO₄, 3.) heating to reflux . In this embodiment, the isomer of formula (V) may be formed, which reduces the yield of the desired compound (I).

As stated above, the compound of formula (II) can be obtained starting from dichloropyridine and hydrazine. Therefore, in a second aspect, the present invention relates to a process as described herein, wherein the compound of the formula (II)

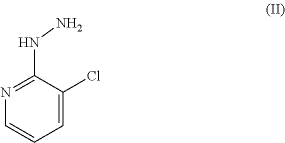

is prepared in step (i) by reacting dichloropyridine compound (VI)
with hydrazine,

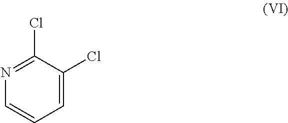

followed by the step (ii) as described herein.

The compounds of of formula (III) may be purchased or may be synthesized according to procedures known in the literature, e.g. Chemistry Letters Vol. 5 (1976) No. 5 p.499-502,

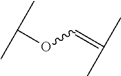

EP744400A2, WO2010/037688. As common in chemical formulas, the bond indicates that the stereogeometry at the double bond is not defined. All stereoisomers are suitable for the reaction.

In the case of R¹=trifluoromethyl, the substance is called “ETFBO” (4-ethoxy-1,1,1-trifluoro-but-3-en-2-one). Therefore, in a further aspect, the present invention relates to a process as described herein, wherein the compound of the formula (III)

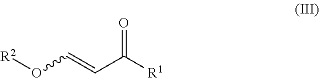

is prepared by reacting the vinyl ether (IIIa)

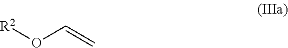

with a reagent selected from trifluoro-/difluoroacetyl chloride, trifluoro-/difluoroacetyl bromide, or trifluoro-/difluoroacetyl anhydride and is provided for step (ii) as described herein as a crude product, optionally together with the primary conversion products of formula (IIIb)

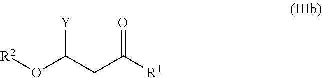

in which Y is chloro or bromo, and R¹ is as defined in any of the preceding claims, followed by the step (ii) as described herein.

This step may be called step (ib).

Therefore, a further aspect of the present invention relates to combinations of the abovementioned process step (ii) with a preceding process step (i) leading to the reactant of formula (II), and/or with a preceding process step (ib), leading to the reactant of formula (III), or with subsequent process steps in which the product of formula (I) is converted to further products, or to a combination of the abovementioned process with preceding and subsequent process steps. The advantages mentioned for the process of step (ii) are also present for the combination of these process steps.

Accordingly, the present invention relates to a process for subsequent reaction of the compounds of formula (I). Derivatives of compounds of formula (I) are e.g. substituted 1-pyridin-2-yl-1H-pyrazole-5-carbonyl compounds of formula (I-A), which are useful in the synthesis of anthranilamide insecticides, especially the carbonyl chlorides. For preparation of substituted 1-pyridin-2-yl-1H-pyrazole-5-carbonylchlorides, a process described in WO 02/070483, WO03/015519, WO 07/043677 and WO 08/130021 has been found to be useful. Especially useful preparation methods are described in WO2013/024007 and in WO2013/076092.

Accordingly, in a further aspect, the present invention relates to a process for preparing a compound of formula (I-A)

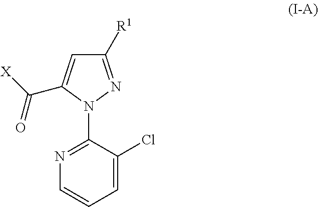

wherein
R¹ is as defined herein;
X is selected from halogen, preferably Cl, OH, O—Mg—Cl, O—Mg—Br, imidazole, —O—CO—R^{x}, —O—CO—OR^{x}, —OSO₂R^{x}, —SR^{y}, in which
R^{x} is independently selected from C₁-C₆-alkyl, trifluoromethyl and phenyl which is optionally substituted with C₁-C₆-alkyl (preferably as o-toluene, m-toluene, p-toluene, o-xylene, m-xylene, p-xylene) or halogen, and
R^{y} is independently selected from C₁-C₆-alkyl and phenyl which is optionally substituted with C₁-C₆-alkyl (preferably as o-toluene, m-toluene, p-toluene, o-xylene, m-xylene, p-xylene) or halogen;

the process comprising:
a) providing the compound of the formula (I) by a process as described herein [step (ii), optionally with a preceding step (i) and/or step (ib)],

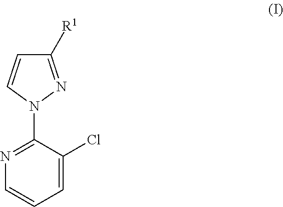

wherein R¹ is as defined above;
b) reacting the compound of formula (I) in a step (iii) to the corresponding carbonyl compound of formula (I-A).

In one embodiment, the invention relates to the process, wherein the carbonyl compound of formula (I-A) is an acid chloride, in which X is Cl.

In a further embodiment, the invention relates to a process as described above, comprising the steps of
iii-a) deprotonating a compound of the formula (I)

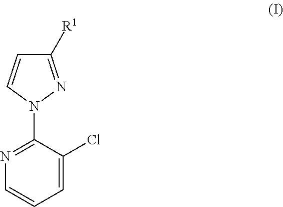

in which R¹ is as defined above,
with a magnesium-organic base having a carbon bound magnesium, or with a magnesium amide having a nitrogen bound magnesium which is derived from a secondary amine, in the presence of a lithium halide, where the base is used in an amount sufficient to achieve at least 80% deprotonation of the compound of formula (I); and
iii-b) subjecting the product obtained in step (iii-a) to a carboxylation by reacting it with a reagent selected from phosgene and carbon dioxide, to obtain a compound of formula (I-A) as defined above.

In a further embodiment, the invention relates to the process as described above, wherein the conversion of a compound of formula (I) to a carbonyl compound of formula (I-A) (step iii) is done in an aprotic organic solvent or aprotic solvent mixture comprising an aprotic solvent having an ether moiety.

The details of the process step (iii), together with preferences and examples, can be found in WO2013/024007 and WO2013/076092,

As said above, the invention relates to combinations of process steps, comprising step (ii). Accordingly, in a further aspect, the present invention relates to a process for preparing an anthranilamide compound of formula (I-B):

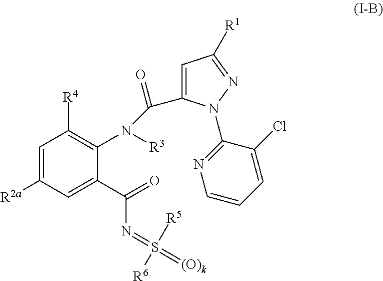

wherein
R¹ is as defined in any of the preceding claims;
R^{2a} is selected from the group consisting of hydrogen, halogen, halomethyl and cyano;
R³ is selected from hydrogen, C₁-C₆ alkyl;
R⁴ is selected from the group consisting of halogen, methyl and halomethyl;
R⁵, R⁶ are selected independently of one another from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
   R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C=S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present;
R^{a} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄ alkoxy;
   phenyl, benzyl, pyridyl and phenoxy, wherein the last four radicals may be unsubstituted, partially or fully halogenated and/or carry 1, 2 or ₃ substituents selected from C₁-C₆-alkyl, C₁-C₆-haloalkyl, C₁-C₆-alkoxy, C₁-C₆-haloalkoxy, (C₁-C₆-alkoxy)carbonyl, C₁-C₆-alkylamino and di-(C₁-C₆-alkyl)amino,
R^{b} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄-alkoxy;
   phenyl, benzyl, pyridyl and phenoxy, wherein the last four radicals may be unsubstituted, partially or fully halogenated and/or carry 1, 2 or ₃ substituents selected from C₁-C₆-alkyl, C₁-C₆-haloalkyl, C₁-C₆-alkoxy, C₁-C₆-haloalkoxy and (C₁-C₆-alkoxy) carbonyl;
R^{c}, R^{d} are, independently from one another and independently of each occurrence, selected from the group consisting of hydrogen, cyano, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄-alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, phenyl, benzyl, pyridyl and phenoxy, wherein the four last mentioned radicals may be unsubstituted, partially or fully halogenated and/or carry 1, 2 or ₃ substituents selected from C₁-C₆-alkyl, C₁-C₆-haloalkyl, C₁-C₆- alkoxy, C₁-C₆ haloalkoxy and (C₁-C₆-alkoxy)carbonyl; or
   R^{c} and R^{d}, together with the nitrogen atom to which they are bound, may form a 3-, 4-, 5-, 6- or 7-membered saturated, partially unsaturated or fully unsaturated heterocyclic ring which may additionally contain 1 or 2 further heteroatoms or heteroatom groups selected from N, O, S, NO, SO and SO₂, as ring members, where the heterocyclic ring may optionally be substituted with halogen, C₁-C₄-haloalkyl, C₁-C₄-alkoxy or C₁-Ca-haloalkoxy;
R^{e} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙ NR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or ₃ substituents selected from C₁-C₆-alkyl, C₁-C₆-haloalkyl, C₁-C₆-alkoxy and C₁-C₆-haloalkoxy; or
   two vicinal radicals Re together form a group ═O, ═CH(C₁-C₄-alkyl), ═C(C₁-C₄-alkyl) C₁-C₄-alkyl, =N(C₁-C₆-alkyl) or =NO(C₁-C₆-alkyl);
R^{f} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, and —C(═NR_{c})NR_{c}R_{d};
k is 0 or 1;
n is 0, 1 or 2;
   or a stereoisomer, salt, tautomer or N-oxide, or a polymorphic crystalline form, a co-crystal or a solvate of a compound or a stereoisomer, salt, tautomer or N-oxide thereof;
   the process comprising
a) providing a compound of the formula (I) by a process as described herein [step (ii), optionally with a preceding step (i) and/or step (ib)],
b) converting the compound of formula (I) to a compound of formula (I-B), optionally via the corresponding carbonyl compound of formula (I-A) as described herein.

In one embodiment, the invention relates to a process for preparing an anthranilamide compound of formula (I-B1):

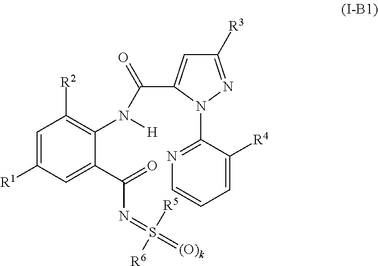

wherein
R¹ is selected from the group consisting of H, F, Cl, Br and CN;
R² is selected from the group consisting of F, Cl, Br, I, CH_{3;}
R³ is selected from the group consisting of Br, Cl, CHF₂, CF₃ and OCH₂F;
R⁴ is Cl or CF3;
R⁵, R⁶ are selected independently of one another from the group consisting of hydrogen, C₁-C₄-alkyl, C₃-C₈-cycloalkyl, or
   R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring,
k is 0 or 1;
   or a stereoisomer, salt, tautomer or N-oxide, or a polymorphic crystalline form, a co-crystal or a solvate of a compound or a stereoisomer, salt, tautomer or N-oxide thereof;
   the process comprising
a) providing a compound of the formula (I) by a process as described herein [step (ii), optionally with a preceding step (i) and/or step (ib)],
b) converting the compound of formula (I) to a compound of formula (I-B), optionally via the corresponding carbonyl compound of formula (I-A) as described herein.

In one embodiment, the invention relates to a process as described above for preparing an anthranilamide compound of formula (I-B1), wherein the compound of formula (I-B1) is selected from the group consisting of the following compounds I-11, I-16, I-21, I-26, I-31:

| | R¹ | R² | R³ | R⁵ | R⁶ | k |
|---|---|---|---|---|---|---|
| I-11 | Cl | CH₃ | CF₃ | C₂H₅ | C₂H₅ | 0 |
| I-16 | Cl | Cl | CF₃ | C₂H₅ | C₂H₅ | 0 |
| I-21 | Cl | CH₃ | CF₃ | CH(CH₃)₂ | CH(CH₃)₂ | 0 |
| I-26 | Cl | Cl | CF₃ | CH(CH₃)₂ | CH(CH₃)₂ | 0 |
| I-31 | Br | Br | CF₃ | C₂H₅ | C₂H₅ | 0 |

In a further aspect, the present invention relates to a process for preparing an anthranilamide compound of formula (I-B) or (1-B1), wherein the process comprises
a) providing a compound of the formula (I) by a process as described herein [step (ii), optionally with a preceding step (i) and/or step (ib)],
b) reacting the compound of formula (I) in a step (iii) to the corresponding carbonyl compound of formula (I-A) as described herein,
c) converting the compound of formula (I-A) in a step (iv) to a compound of formula (I-B) as described herein.

In a further aspect, the present invention relates to a process for preparing an anthranilamide compound of formula (I-B) as described herein, wherein the process step (iv) in c) comprises
iv) reacting the compound of the formula (I-A) as described herein with a compound of the formula (V)

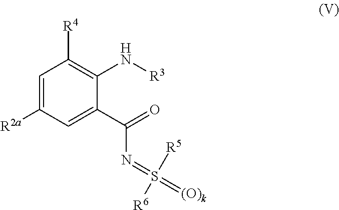

in which the variables R²a, R³, R⁴, R⁵, R⁶ and k are each as defined in any of claim 14 or 16,
in the presence of a base, to obtain a compound of the formula (I-B) as defined herein.

In a further aspect, the present invention relates to a process for preparing an anthranilamide compound of formula (I-B), wherein in the compound of formula (I-B)
R¹ is as defined herein,

R²a is Cl, Br, cyano;
R³ is hydrogen, methyl;
R⁴ is methyl, Cl, Br;
R⁵ and R⁶ are identical and selected from methyl, ethyl, isopropyl;
k is 0.

In the context of the present invention, the terms used generically are each defined as follows:

The prefix C.-C_{y} refers in the particular case to the number of possible carbon atoms.

The term “halogen” denotes in each case fluorine, bromine, chlorine or iodine, in particular fluorine, chlorine or bromine.

The term “partially or fully halogenated” will be taken to mean that 1 or more, e.g. 1, 2, 3, 4 or 5 or all of the hydrogen atoms of a given radical have been replaced by a halogen atom, in particular by fluorine or chlorine.

The term “alkyl” as used herein (and in the alkyl moieties of other groups comprising an alkyl group, e.g. alkoxy, alkylcarbonyl, alkylthio, alkylsulfinyl, alkylsulfonyl and alkoxyalkyl) denotes in each case a straight-chain or branched alkyl group having usually from 1 to 10 carbon atoms, frequently from 1 to 6 carbon atoms, preferably 1 to 4 carbon atoms and in particular from 1 to ₃ carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, n-octyl, 1-methyloctyl, 2-methylheptyl, 1-ethylhexyl, 2-ethylhexyl, 1,2-dimethylhexyl, 1-propylpentyl and 2-propylpentyl.

The term “alkylene” (or alkanediyl) as used herein in each case denotes an alkyl radical as defined above, wherein one hydrogen atom at any position of the carbon backbone is replaced by one further binding site, thus forming a bivalent moiety.

The term “haloalkyl” as used herein (and in the haloalkyl moieties of other groups comprising a haloalkyl group, e.g. haloalkoxy and haloalkylthio) denotes in each case a straight- chain or branched alkyl group having usually from 1 to 10 carbon atoms, frequently from 1 to 6 carbon atoms, wherein the hydrogen atoms of this group are partially or totally replaced with halogen atoms. Preferred haloalkyl moieties are selected from C₁-C₄-haloalkyl, more preferably from C₁-C₂-haloalkyl, more preferably from halomethyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “fluoroalkyl”, as used herein (and in the fluoroalkyl units of fluoroalkoxy, fluoroalkylthio, fluoroalkylsulfinyl and fluoroalkylsulfonyl) denotes in each case straight-chain or branched alkyl groups having usually from 1 to 10 carbon atoms, frequently from 1 to 6 carbon atoms and in particular 1 to 4 carbon atoms, wherein the hydrogen atoms of this group are partially or totally replaced with fluorine atoms. Examples thereof are fluoromethyl, difluoromethyl, trifluoromethyl, 1-fluoroethyl, 2-fluoroethyl, 2,2-difluoroethyl, 2,2,2-trifluoroethyl, pentafluoroethyl, 3,3,3-trifluoroprop-1-yl, 1,1,1-trifluoroprop-2-yl, heptafluoroisopropyl, 1-fluorobutyl, 2-fluorobutyl, 3-fluorobutyl, 4-fluorobutyl, 4,4,4-trifluorobutyl, fluoro-tert-butyl and the like.

The term “cycloalkyl” as used herein (and in the cycloalkyl moieties of other groups comprising a cycloalkyl group, e.g. cycloalkoxy and cycloalkylalkyl) denotes in each case a mono- or bicyclic cycloaliphatic radical having usually from 3 to 10 carbon atoms, 3 to 8 carbon atoms or 3 to 6 carbon atoms, such as cyclopropyl, cyclobutyl, cyclopentyl, cyclohexyl, cycloheptyl, cyclooctyl, bicyclo[2.1.1]hexyl, bicyclo[3.1.1]heptyl, bicyclo[2.2.1]heptyl, and bicyclo[2.2.2]octyl.

The term “halocycloalkyl” as used herein (and in the halocycloalkyl moieties of other groups comprising an halocycloalkyl group, e.g. halocycloalkylmethyl) denotes in each case a mono- or bicyclic cycloaliphatic radical having usually from 3 to 10 carbon atoms, 3 to 8 carbon atoms or 3 to 6 carbon atoms, wherein at least one, e.g. 1, 2, 3, 4 or 5 of the hydrogen atoms are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlorocyclopentyl and the like.

The term “fluorocylcoalkyl” as used herein, denotes a halocycloalkyl radical, as defined above, wherein the one or more halogen atoms are fluorine atoms.

The term “alkenyl” as used herein denotes in each case a singly unsaturated hydrocarbon radical having usually 2 to 10, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “alkenylene” (or alkenediyl) as used herein in each case denotes an alkenyl radical as defined above, wherein one hydrogen atom at any position of the carbon backbone is replaced by one further binding site, thus forming a bivalent moiety.

The term “haloalkenyl” as used herein, which may also be expressed as “alkenyl which may be substituted by halogen”, and the haloalkenyl moieties in haloalkenyloxy, haloalkenylcarbonyl and the like refers to unsaturated straight-chain or branched hydrocarbon radicals having 2 to 10 (“C₂-C₁₀-haloalkenyl”) or 2 to 6 (“C₂-C₆-haloalkenyl”) carbon atoms and a double bond in any position, where some or all of the hydrogen atoms in these groups are replaced by halogen atoms as mentioned above, in particular fluorine, chlorine and bromine, for example chlorovinyl, chloroallyl and the like. The term “fluoroalkenyl” as used herein, denotes a haloalkenyl radical, as defined above, wherein the one or more halogen atoms are fluorine atoms.

The term “alkynyl” as used herein denotes unsaturated straight-chain or branched hydrocarbon radicals having usually 2 to 10, frequently 2 to 6, preferably 2 to 4 carbon atoms and one or two triple bonds in any position, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “alkynylene” (or alkynediyl) as used herein in each case denotes an alkynyl radical as defined above, wherein one hydrogen atom at any position of the carbon backbone is replaced by one further binding site, thus forming a bivalent moiety. The term “haloalkynyl” as used herein, which is also expressed as “alkynyl which may be substituted by halogen”, refers to unsaturated straight-chain or branched hydrocarbon radicals having usually 3 to 10 carbon atoms, frequently 2 to 6, preferably 2 to 4 carbon atoms, and one or two triple bonds in any position (as mentioned above), where some or all of the hydrogen atoms in these groups are replaced by halogen atoms as mentioned above, in particular fluorine, chlorine and bromine.

The term “alkoxy” as used herein denotes in each case a straight-chain or branched alkyl group usually having from 1 to 10 carbon atoms, frequently from 1 to 6 carbon atoms, preferably 1 to 4 carbon atoms, which is bound to the remainder of the molecule via an oxygen atom. Examples of an alkoxy group are methoxy, ethoxy, n-propoxy, iso-propoxy, n-butyloxy, 2-butyloxy, iso-butyloxy, tert-butyloxy, and the like.

The term “haloalkoxy” as used herein denotes in each case a straight-chain or branched alkoxy group, as defined above, having from 1 to 10 carbon atoms, frequently from 1 to 6 carbon atoms, preferably 1 to 4 carbon atoms, preferably 1 to ₃ carbon atoms, wherein the hydrogen atoms of this group are partially or totally replaced with halogen atoms, in particular fluorine atoms. Preferred haloalkoxy moieties include C₁-Ca-haloalkoxy, in particular halomethoxy, and also in particular C₁-C₂-fluoroalkoxy, such as fluoromethoxy, difluoromethoxy, trifluoromethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoro-ethoxy, 2,2-dichloro-2-fluorethoxy, 2,2,2-trichloroethoxy, pentafluoroethoxy and the like.

The term “alkoxy-alkyl” as used herein denotes in each case alkyl usually comprising 1 to 6 carbon atoms, preferably 1 to 4 carbon atoms, wherein 1 carbon atom carries an alkoxy radical usually comprising 1 to 10, frequently 1 to 6, in particular 1 to 4, carbon atoms as defined above. Examples are CH₂OCH₃, CH₂—OC₂H₅, n-propoxymethyl, CH₂—OCH(CH₃)₂, n-butoxymethyl, (1-methylpropoxy)-methyl, (2-methylpropoxy)methyl, CH₂—OC(CH₃)₃, 2-(methoxy)ethyl, 2-(ethoxy)ethyl, 2-(n-propoxy)-ethyl, 2-(1-methylethoxy)-ethyl, 2-(n-butoxy)ethyl, 2-(1-methylpropoxy)-ethyl, 2-(2-methylpropoxy)-ethyl, 2-(1,1-dimethylethoxy)-ethyl, 2-(methoxy)-propyl, 2-(ethoxy)-propyl, 2-(n-propoxy)-propyl, 2-(1-methylethoxy)-propyl, 2-(n-butoxy)-propyl, 2-(1-methylpropoxy)-propyl, 2-(2-methylpropoxy)-propyl, 2-(1,1-dimethylethoxy)-propyl, 3-(methoxy)-propyl, 3-(ethoxy)-propyl, 3-(n-propoxy)-propyl, 3-(1-methylethoxy)-propyl, 3-(n-butoxy)-propyl, 3-(1-methylpropoxy)-propyl, 3-(2-methylpropoxy)-propyl, 3-(1,1-dimethylethoxy)-propyl, 2-(methoxy)-butyl, 2-(ethoxy)-butyl, 2-(n-propoxy)-butyl, 2-(1-methylethoxy)-butyl, 2-(n-butoxy)-butyl, 2-(1-methylpropoxy)-butyl, 2-(2-methyl-propoxy)-butyl, 2-(1,1-dimethylethoxy)-butyl, 3-(methoxy)-butyl, 3-(ethoxy)-butyl, 3-(n-propoxy)-butyl, 3-(1-methylethoxy)-butyl, 3-(n-butoxy)-butyl, 3-(1-methylpropoxy)-butyl, 3-(2-methylpropoxy)-butyl, 3-(1,1-dimethylethoxy)-butyl, 4-(methoxy)-butyl, 4-(ethoxy)-butyl, 4-(n-propoxy)-butyl, 4-(1-methylethoxy)-butyl, 4-(n-butoxy)-butyl, 4-(1-methylpropoxy)-butyl, 4-(2-methylpropoxy)-butyl, 4-(1,1-dimethylethoxy)-butyl and the like.

The term “fluoroalkoxy-alkyl” as used herein denotes in each case alkyl as defined above, usually comprising 1 to 6 carbon atoms, preferably 1 to 4 carbon atoms, wherein 1 carbon atom carries an fluoroalkoxy radical as defined above, usually comprising 1 to 10, frequently 1 to 6, in particular 1 to 4, carbon atoms as defined above. Examples are fluoromethoxymethyl, difluoromethoxymethyl, trifluoromethoxymethyl, 1-fluoroethoxymethyl, 2-fluoroethoxymethyl, 1,1-difluoroethoxymethyl, 1,2-difluoroethoxymethyl, 2,2-difluoroethoxymethyl, 1,1,2-trifluoroethoxymethyl, 1,2,2-trifluoroethoxymethyl, 2,2,2-trifluoroethoxymethyl, pentafluoroethoxymethyl, 1-fluoroethoxy-1-ethyl, 2-fluoroethoxy-1-ethyl, 1,1-difluoroethoxy-1-ethyl, 1,2-difluoroethoxy-1-ethyl, 2,2-difluoroethoxy-1-ethyl, 1,1,2-trifluoroethoxy-1-ethyl, 1,2,2-trifluoroethoxy-1-ethyl, 2,2,2-trifluoroethoxy-1-ethyl, pentafluoroethoxy-1-ethyl, 1-fluoroethoxy-2-ethyl, 2-fluoroethoxy-2-ethyl, 1,1-difluoroethoxy-2-ethyl, 1,2-difluoroethoxy-2-ethyl, 2,2-difluoroethoxy-2-ethyl, 1,1,2-trifluoroethoxy-2-ethyl, 1,2,2-trifluoroethoxy-2-ethyl, 2,2,2-trifluoroethoxy-2-ethyl, pentafluoroethoxy-2-ethyl, and the like.

The term “alkylthio” (also alkylsulfanyl or alkyl-S-)” as used herein denotes in each case a straight-chain or branched saturated alkyl group as defined above, usually comprising 1 to 10 carbon atoms, frequently comprising 1 to 6 carbon atoms, preferably 1 to 4 carbon atoms, which is attached via a sulfur atom at any position in the alkyl group. Examples are methylthio, ethylthio, n-propylthio, iso-propylthio, n-butylthio, 2-butylthio, iso-butylthio, tert-butylthio, and the like.

The term “haloalkylthio” as used herein refers to an alkylthio group as defined above wherein the hydrogen atoms are partially or fully substituted by fluorine, chlorine, bromine and/or iodine. Examples are fluoromethylthio, difluoromethylthio, trifluoromethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoro-ethylthio, 2,2-dichloro-2-fluorethylthio, 2,2,2-trichloroethylthio, pentafluoroethylthio and the like

The terms “alkylsulfinyl” and “S(O)ₙ-alkyl” (wherein n is 1) are equivalent and, as used herein, denote an alkyl group, as defined above, attached via a sulfinyl [S(O)] group. For example, the term “C₁-C₆-alkylsulfinyl” refers to a C₁-C₆-alkyl group, as defined above, attached via a sulfinyl [S(O)] group. Examples are methylsulfinyl, ethylsulfinyl, n-propylsulfinyl, 1-methylethylsulfinyl (isopropylsulfinyl), butylsulfinyl, 1-methylpropylsulfinyl (sec-butylsulfinyl), 2-methylpropylsulfinyl (isobutylsulfinyl), 1,1-dimethylethylsulfinyl (tert-butylsulfinyl), pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl.

The terms “alkylsulfonyl” and “S(O)ₙ-alkyl” (wherein n is 2) are equivalent and, as used herein, denote an alkyl group, as defined above, attached via a sulfonyl [S(O)2] group. For example, the term “C₁-C₆-alkylsulfonyl” refers to a C₁-C₆-alkyl group, as defined above, attached via a sulfonyl [S(O)2] group. Examples are methylsulfonyl, ethylsulfonyl, n-propylsulfonyl, 1-methylethylsulfonyl (isopropylsulfonyl), butylsulfonyl, 1-methylpropylsulfonyl (sec-butylsulfonyl), 2-methylpropylsulfonyl (isobutylsulfonyl), 1,1-dimethylethylsulfonyl (tert-butylsulfonyl), pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl.

The term “alkylamino” as used herein denotes in each case a group —NHR, wherein R is a straight-chain or branched alkyl group usually having from 1 to 6 carbon atoms, preferably 1 to 4 carbon atoms. Examples of an alkylamino group are methylamino, ethylamino, n-propylamino, isopropylamino, n-butylamino, 2-butylamino, iso-butylamino, tert-butylamino, and the like.

The term “dialkylannino” as used herein denotes in each case a group-NRR′, wherein R and independently of each other, are a straight-chain or branched alkyl group each usually having from 1 to 6 carbon atoms, preferably 1 to 4 carbon atoms. Examples of a dialkylamino group are dimethylamino, diethylamino, dipropylamino, dibutylamino, methyl-ethyl-amino, methyl-propyl-amino, methyl-isopropylamino, methyl-butyl-amino, methyl-isobutyl-amino, ethyl- propyl-amino, ethyl-isopropylamino, ethyl-butyl-amino, ethyl-isobutyl-amino, and the like.

The suffix “-carbonyl” in a group denotes in each case that the group is bound to the remainder of the molecule via a carbonyl C═O group. This is the case e.g. in alkylcarbonyl, haloalkylcarbonyl, alkoxycarbonyl and haloalkoxycarbonyl.

The term “aryl” as used herein refers to a mono-, bi- or tricyclic aromatic hydrocarbon radical having 6 to 14 carbon atoms. Examples thereof comprise phenyl, naphthyl, fluorenyl, azulenyl, anthracenyl and phenanthrenyl. Aryl is preferably phenyl or naphthyl and especially phenyl.

The term “3-, 4-, 5-, 6-, 7- or 8-membered saturated carbocyclic ring” as used herein refers to carbocyclic rings, which are monocyclic and fully saturated. Examples of such rings include cyclopropane, cyclobutane, cyclopentane, cyclohexane, cycloheptane, cyclooctane and the like.

The terms “3-, 4-, 5-, 6-, 7- or 8-membered partially unsaturated carbocyclic ring” and “5- or 6-membered partially unsaturated carbocyclic ring” refer to carbocyclic rings, which are monocyclic and have one or more degrees of unsaturation. Examples of such rings include include cyclopropene, cyclobutene, cyclopentene, cyclohexene, cycloheptene, cyclooctene and the like.

The term “3-, 4-, 5-, 6- or 7-membered saturated, partially unsaturated or completely unsaturated heterocyclic ring containing 1, 2 or ₃ heteroatoms or heteroatom groups selected from N, O, S, NO, SO and SO₂, as ring members” [wherein “completely/fully unsaturated” includes also “aromatic”] as used herein denotes monocyclic radicals, the monocyclic radicals being saturated, partially unsaturated or fully unsaturated (including aromatic). The heterocyclic ring may be attached to the remainder of the molecule via a carbon ring member or via a nitrogen ring member.

Examples of a 3-, 4-, 5-, 6- or 7-membered saturated heterocyclic ring include: oxiranyl, aziridinyl, azetidinyl, tetrahydrofuran-2-yl, tetrahydrofuran-3-yl, tetrahydrothien-2-yl, tetrahydrothien-3-yl, pyrrolidin-2-yl, pyrrolidin-3-yl, pyrazolidin-3-yl, pyrazolidin-4-yl, pyrazolidin-5-yl, imidazolidin-2-yl, imidazolidin-4-yl, oxazolidin-2-yl, oxazolidin-4-yl, oxazolidin-5-yl, isoxazolidin-3-yl, isoxazolidin-4-yl, isoxazolidin-5-yl, thiazolidin-2-yl, thiazolidin-4-yl, thiazolidin-5-yl, isothiazolidin-3-yl, isothiazolidin-4-yl, isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2-tetrahydropyranyl, 4-tetrahydropyranyl, 1,3-dioxan-5-yl, 1,4-dioxan-2-yl, piperidin-2-yl, piperidin-3-yl, piperidin-4-yl, hexahydropyridazin-3-yl, hexahydropyridazin-4-yl, hexahydropyrimidin-2-yl, hexahydropyrimidin-4-yl, hexahydropyrimidin-5-yl, piperazin-2-yl, 1,3,5-hexahydrotriazin-2-yl and 1,2,4-hexahydrotriazin-3-yl, morpholin-2-yl, morpholin-3-yl, thiomorpholin-2-yl, thiomorpholin-3-yl, 1-oxothiomorpholin-2-yl, 1-oxothiomorpholin-3-yl, 1,1-dioxothiomorpholin-2-yl, 1,1-dioxothiomorpholin-3-yl, azepan 1,2,3 or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of a 3-, 4-, 5-, 6- or 7-membered partially unsaturated heterocyclic ring include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin 1,2,3,4,5,6or -7-yl, 3,4,5,6-tetrahydro[2H]azepin 2,3,4,5,6or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-1-, -2-, -3-, -4-, -5-, -6- or -7-yl, 2,3,4,7-tetrahydro [1H]oxepin-1-, -2-, -3-, -4-, -5-, -6- or -7-yl, 2,3,6,7-tetrahydro[1H]oxepin-1-, -2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl. A 3-, 4-, 5-, 6- or 7-membered completely unsaturated (including aromatic) heterocyclic ring is e.g. a 5- or 6-membered fully unsaturated (including aromatic) heterocyclic ring. Examples are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 4-isoxazolyl, 2-thiazolyl, 4-thiazolyl, 5-thiazolyl, 4-isothiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

The term “a 3-, 4-, 5-, 6-, 7- or 8-membered saturated or partially unsaturated carbocyclic or heterocyclic ring containing 1, 2 or ₃ heteroatoms or heteroatom groups selected from N, O, S, NO, SO and SO₂, as ring members” as used herein denotes a saturated or unsaturated 3- to 8-membered ring system which optionally contains 1 to ₃ heteroatoms selected from N, O, S, NO, SO and SO₂, as defined above, with the exception of the completely unsaturated ring systems.

### PREFERENCES

Regarding reaction conditions and preferences, details of the process steps (iii) and (iv) may be found in WO2013/076092, along with details of how the sulfimine moiety may be introduced in the compounds of formula (I-B).
The remarks made below concerning preferred embodiments of the variables of the compounds of the formulae (I), (I-A), (I-B), (III) and (V) are valid on their own as well as preferably in combination with each other concerning the compounds of formula (I), (I-A) and (I-B) as well as concerning the methods according to the invention.
In one embodiment of the invention, R¹ is CF3. Especially, in the compounds of the formulae (I), (I-A), (I-B), (III), (IIIb) and (IV), and the processes related to them, R¹ is CF₃.

In a further embodiment, R¹ is CHF₂. Especially, in the compounds of the formulae (I), (I-A), (I-B), (III), (IIIb) and (IV), and the processes related to them, R¹ is CHF₂.

In the compounds of the formulae (I-B) and (V), Rea is hydrogen, halogen, halomethyl or cyano , preferably, Rea is CI or Br or cyano, most preferably Cl.

R⁴ is selected from the group consisting of halogen, methyl and halomethyl; preferably from methyl, Cl, Br; most preferably methyl.

In the compounds of the formulae (I-B) and (V), R³ is hydrogen or methyl, preferably hydrogen.

In the compounds of the formulae (I-B) and (V), t is preferably 0.

In the compounds of the formulae (I-B) and (V), wherein t is O, R⁵ and R⁶ are preferably, independently of each other, selected from hydrogen, C₁-C₆-alkyl, C₁-C₆-haloalkyl, C₃-C₆-cycloalkyl, C₃-C₆-halocycloalkyl, C₂-C₄-alkenyl, C₂-C₄-haloalkenyl, wherein the six last radicals may optionally be substituted by one or more radicals R^{a};

or R⁶ and R⁷ together represent a C₄-C₅-alkylene or C₄-C₅-alkenylene chain forming together with the sulfur atom to which they are attached a 5- or 6-membered saturated or partially unsaturated ring, wherein one of the CH₂ groups in the C₄-C₅-alkylene chain or one of the CH₂ or CH groups in the C₄-C₅-alkenylene chain may be replaced by a group independently selected from O, S and N and NH, and wherein the carbon and/or nitrogen atoms in the C₄-C₅-alkylene or C₄-C₅-alkenylene chain may be substituted with 1 or 2 substituents independently selected from halogen, cyano, C₁-C₄-alkyl, C₁-C₄-haloalkyl, C₁-C₄-alkoxy, C₁-C₄-haloalkoxy.

More preferably R⁵ and R⁶ are independently selected from C₁-C₆-alkyl, C₁-C₆-haloalkyl, or R⁵ and R⁶ together represent a C₄-C₅-alkylene chain forming together with the sulfur atom to which they are attached a 5- or 6-membered ring. Particularly preferred R⁵ and R⁶ are each C₁-C₆-alkyl, or together represent a C₄-C₅-alkylene chain forming together with the sulfur atom to which they are attached a 5- or 6-membered ring. More preferably R⁵ and R⁶ are independently selected from C₁-C₄-alkyl, C₁Ca-haloalkyl, or R⁵ and R⁶ together represent a C₄-C₅-alkylene chain forming together with the sulfur atom to which they are attached a 5- or 6-membered ring. Particularly preferred R⁵ and R⁶ are each C₁-C₄-alkyl, or together represent a C₄-C₅-alkylene chain forming together with the sulfur atom to which they are attached a 5- or 6-membered ring. Particularly preferred, when t is 0, R⁵ and R⁶ are selected independently of one another from C₁-C₆-alkyl, or R⁵ and R⁶ together represent a C₃-C₆-alkylene chain forming together with the sulfur atom to which they are attached a 4-, 5-, 6- or 7-membered saturated ring. Specifically R⁵ and R⁶ are each methyl, isopropyl or ethyl, or together represent a butylene chain forming together with the sulfur atom to which they are attached a 5-membered ring.

In the compounds of the formulae (I-B) and (V), wherein t is 1, the preferred meanings of R⁵ and R⁶ are the preferred meanings as described above in the compounds of the formulae (VI) and (VII), wherein t is 0.

In this context, the variables R^{a}, R^{b}, R^{c}, R^{d}, R^{b1}, R^{c1}, R^{d1}, R^{e}, R^{f}, R^{g}, R^{h}, Rⁱ, m and n, independently of each other, preferably have one of the following meanings:

R^{a} is selected from C₁-C₄-alkyl, C₁-C₄-fluoroalkyl, C₃-C₆-cycloalkyl, C₃-C₆-fluorocycloalkyl, C₂-C₄-alkenyl, C₂-C₄-fluoroalkenyl, C₁-C₄-alkoxy, C₁-C₄-alkylthio, amino, di-(C₁-C₄-alkyl)-amino, phenyl and a 5- or 6-membered saturated, partially unsaturated or completely unsaturated heterocyclic ring containing 1 or 2 heteroatoms selected from N, O and S, as ring members, where phenyl and the heterocyclic ring may be substituted by 1, 2 or ₃ radicals selected from C₁-C₄-alkyl, C₁-C₄-fluoroalkyl, C₅-C₆-cycloalkyl and C₅-C₆-fluorocycloalkyl.

More preferably R^{a} is selected from C₁-C₄-alkyl and C₁-C₄-fluoroalkyl, C₁-C₄-alkoxy, di-(C₁-C₄-alkyl)-amino, phenyl and a 5- or 6-membered saturated, partially unsaturated or completely unsaturated heterocyclic ring containing 1 or 2 heteroatoms selected from N, O and S, as ring members, and in particular selected from C₁-C₃-alkyl and C₁-C₂-fluoroalkyl and C₁-C₂-alkoxy.

R^{b} is selected from C₁-C₄-alkyl, C₁Cₐ-fluoroalkyl, C₅-C₆-cycloalkyl, C₅-C₆-fluorocycloalkyl, C₁-C₄-alkoxy-C₁-C₄-alkyl, C₁-C₄-fluoroalkoxy-C₁-C₄-alkyl, phenyl-C₁-C₄-alkyl, phenoxy-C₁-C₄-alkyl and pyridyl-C₁-C₄-alkyl, wherein phenyl and pyridyl in the three last mentioned radicals may optionally carry 1 or 2 radicals selected from halogen, substituents C₁-C₄-alkyl, C₁-C₂-fluoroalkyl, C₁-C₄-alkoxy and C₁-C₂-fluoroalkoxy.

More preferably R^{b} is selected from C₁-C₄-alkyl, C₁-C₄-fluoroalkyl and benzyl, and in particular selected from C₁-C₃-alkyl, C₁-C₂-fluoroalkyl and benzyl.

R^{c}, R^{d} are, independently from one another and independently of each occurrence, selected from C₁-C₄-alkyl, C₁-C₄-fluoroalkyl, C₅-C₆-cycloalkyl, C₅-C₆-fluorocycloalkyl, wherein the four last mentioned radicals may optionally carry 1 or 2 radicals selected from C₁-C₄-alkoxy, C₁-C₄-fluoroalkoxy, C₁-C₄-alkylthio, C₁-C₄-fluoroalkylthio, phenyl, benzyl, pyridyl and phenoxy, wherein the four last mentioned radicals may carry 1 or 2 substituents selected from halogen, C₁-C₄-alkyl, C₁-C₂-fluoroalkyl, C₁-C₄-alkoxy and C₁-C₂-fluoroalkoxy; or R^{c} and R^{d}, together with the nitrogen atom to which they are bound, form a 5- or 6-membered saturated, partly unsaturated or completely unsaturated heterocyclic ring which may contain 1 further heteroatom selected from N, 0 and S as ring members, where the heterocyclic ring may carry 1 or 2 substituents selected from halogen, C₁-C₄-alkyl and C₁-C₄-fluoroalkyl. More preferably R^{c}, R^{d} are, independently from one another and independently of each occurrence, selected from C₁-C₄-alkyl, C₁-C₄-fluoroalkyl and benzyl, or R^{c} and R^{d}, together with the nitrogen atom to which they are bound, form a 5- or 6-membered saturated or partly unsaturated heterocyclic ring. In particular, R^{c}, R^{d} are, independently from one another and independently of each occurrence, C₁-C₃-alkyl, C₁-C₂-fluoroalkyl, benzyl, or together with the nitrogen atom to which they are bound form a pyrrolidine or a piperidine ring.

R^{b1} is hydrogen or has one of the preferred meanings given for R^{c}.

R^{c1} is hydrogen or has one of the preferred meanings given for R^{c}.

R^{d1} is hydrogen or has one of the preferred meanings given for R^{d}.

R^{e} is selected from halogen, C₁-C₄-alkyl, C₁-C₄-fluoroalkyl, C₂-C₄-alkenyl, C₂-C₄-fluoroalkenyl, where the four last mentioned radicals may optionally carry 1 or 2 radicals selected from C₁-C₂-alkoxy; C₁-C₄-alkoxy, C₁-C₄-fluoroalkoxy, phenyl, benzyl, pyridyl and phenoxy, wherein the four last mentioned radicals may carry 1 or 2 substituents selected from halogen, C₁-C₂-alkyl and C₁-C₂-fluoroalkyl.

More preferably R^{e} is selected from C₁-C₄-alkyl, C₁-C₄-fluoroalkyl, C₁-C₄-alkoxy and C₁-C₄-fluoroalkoxy, and in particular from C₁-C₃-alkyl, C₁-C₂-fluoroalkyl, C₁-C₂-alkoxy, C₁-C₂-fluoroalkoxy.

R^{f}, R^{g} are, independently of each other and independently of each occurrence, selected from C₁-C₄-alkyl, C₅-C₆-cycloalkyl, C₁-C₂-alkoxy-C₁-C₂-alkyl, phenyl and benzyl.

More preferably R^{f}, R^{g} are, independently of each other and independently of each occurrence, selected from C₁-C₄-alkyl, C₅-C₆-cycloalkyl, benzyl and phenyl, and in particular from C₁-C₃-alkyl, benzyl and phenyl.

R^{h}, Rⁱ are, independently from one another and independently of each occurrence, selected from hydrogen, halogen, C₁-C₄-alkyl, C₁-C₄-fluoroalkyl, C₅-C₆-cycloalkyl, C₅-C₆-fluorocycloalkyl, where the four last mentioned radicals may optionally carry 1 or 2 radicals selected from C₁-C₃-alkyl and C₁-C₃-fluoroalkyl; C₁-C₄-alkoxy, C₁-C₄-fluoroalkoxy, phenyl, pyridyl and phenoxy.

More preferably R^{h}, Rⁱ are, independently of each other and independently of each occurrence, selected from hydrogen, C₁-C₃-alkyl and C₁-C₂-fluoroalkyl.

m is 1 or 2, wherein, in the case of several occurrences, m may be identical or different. More preferably m is 2.

n is 1 or 2, wherein, in the case of several occurrences, n may be identical or different. More preferably n is 2.

### EXAMPLES

The compounds can be characterized e.g. by High Performance Liquid Chromatography, by ¹H-/¹³C-NMR and/or by their melting or boiling points. The following analytical procedures were employed:

Analytical HPLC column: Zorbax Eclipse XDB-C_{18 1.8} μm 50*4.6 mm von Agilent®Elution: acetonitrile +0.1 Vol % H₃PO₄ /water+0.1 Vol % H₃PO₄ in a ratio of from 20:80 to 80:20 in 11 minutes at 40° C., UV detection at 212 nm.

¹H-/¹³C-NMR. The signals are characterized by chemical shift (ppm) vs. tetramethylsilane, by their multiplicity and by their integral (relative number of hydrogen atoms given). The following abbreviations are used to characterize the multiplicity of the signals: m=multiplett, q=quartett, t=triplett, d=doublet and s=singulett.

m.p. is melting point, b.p. is boiling point.
Room temperature means usually 20-25° C.

### Starting Materials

2,3-Dichloropyridine was purchased from Aldrich.
(3-Chloro-2-pyridyl)hydrazine (II) was prepared according to JOC 35 S.810 (1970) from reaction of 2,3-dichloropyridine with hydrazine hydrate. Purity was from 95,9 wt-% to 99.3 wt-% and usually is indicated in the example description.
ETFBO (4-ethoxy-1,1,1-trifluoro-but-3-en-2-one) was prepared according to Chem. Lett., pp. 499-502, 1976, or may be purchased e.g. from Solvay.

### Comparison Examples

### Comparison Example C1: see Europ.J.Med.Chem 2003_38_S157ff

a) Preparation of (3-Chloro-2-pyridyl)hydrazine hydrochloride

20.5 g (3-Chloro-2-pyridyl)hydrazine was charged together with 210 g toluene in a 500 ml flask. After addition of 19 g conc. hydrochloric acid the mixture was heated to reflux and 16.5 g water were removed by azeotropic distillation. The solid product was isolated by filtration and dried at 50° C./10 mbar. 23.8 g of a yellowish solid were obtained.
1H-NMR (400 MHz, DMSO): δ/ppm=7.03 (m, 1H), 7.91 (m, 1H), 8.21 (m, 1H), 9.38 (s, NH), 10.45 s broad, NH₃⁺)
b) reaction of (3-Chloro-2-pyridyl)hydrazine hydrochloride with ETFBO in ethanole
10 g (3-Chloro-2-pyridyl)hydrazine hydrochloride was charged with 105 g ethanol in a 250 ml flask. Then 9.4 g ETFBO were added at room temperature (21° C.). The reaction mixture was heated to reflux (78° C.). HPLC control of the homogeneous orange reaction mixture after 4 h showed the formation of 3-chloro-2-[3-(trifluoromethyppyrazol-1-yl]pyridine and the isomer, 3-chloro-2-[5-(trifluoromethyppyrazol-1-yl]pyridine in a ratio of 4:1. After evaporation of the solvent 12,9 g of a brown oil was obtained. The yield of the desired 3-chloro-2-[3-(trifluoromethyppyrazol-1-yl]pyridine in the evaporation residue was calculated by quantitative HPLC analysis to be 64%, the yield of the undesired isomer was determined to be 16%.

### Comparison Example C_{2:} see Tetrahedron 67 (2011) p. 5663

20.5 g (3-Chloro-2-pyridyl)hydrazine was charged with 210 g ethanol in a 500 ml flask. Then 25.3 g ETFBO were added at room temperature (21° C.). The reaction mixture was heated to reflux (78° C.). HPLC control of the homogeneous orange reaction mixture after 4 and 14 h showed only traces of the desired 3-Chloro-2-[3-(trifluoromethyl)-1H-pyrazol-1yl]pyridine and its isomer 3-Chloro-2-[5-(trifluoromethyl)-1H-pyrazol-1yl]pyridine. The main product of the reaction was isolated as a solid in a quantity of 11.5 g after trituration of the evaporation residue with 20 ml of diisopropylether. The product was characterized as 8-Chloro-[1,2,4]triazole[4.3-a]pyridine by NMR-analyis.

### Product Characterization:

13C-NMR (125 MHz, DMSO): δ/ppm=113.55 (d), 119.54 (s), 124.32 (d), 127.15 (d), 138.12 (d), 146.40(s) 1H-NMR (400 MHz, DMSO): δ/ppm =6.98 (dd, 1H), 7.62 (d, 1H), 8.58(d, 1H),9,40 (s,1H)

### EXAMPLES OF THE PRESENT INVENTION

### Example 1

20.7 g (3-Chloro-2-pyridyl)hydrazine (99.3 wt-%) were suspended in 210 g toluene. Then, 25.2 g ETFBO were added at room temperature (21° C.). Upon warming to 29° C., an orange solution was formed (HPLC control: contains 2-(3-chloro-2-pyridyl)-3-(trifluoromethyl)-4H-pyrazol-3-ole) Subsequently, at 25° C., 2.1 g concentrated sulfuric acid were added, and the solution was heated under reflux for 21 hours. After cooling to 25° C., a small phase of reaction water was observed. The organic phase contained (HPLC control) the isomers 3-chloro-2-[3-(trifluoromethyl)-1H-pyrazol-1yl]pyridine and 3-Chloro-2-[5-(trifluoromethyl)-1H-pyrazol-1yl]pyridine in a ratio of 5.2:1.

### Characterization of the Products:

a) 3-Chloro-2-[3-(trifluoromethyl)-1H-pyrazol-1yl]pyridine
The compound was isolated as crude product and characterized by NMR spectroscopy and found to be identical to the compound prepared according to known literature (Bioorg. Med. Chem. Lett. 15 (2005) 4898-4906).
¹³C-NMR (125 MHz, DMSO): δ/ppm=107.10 (d), 121.34 (q, ¹J (C,F)=268.6 Hz), 125.35 (s), 134.14 (d), 126.57 (d), 141.02(d), 142.70 (q, ²J (C,F)=37.5 Hz), 147.19 (s), 147.56 (d) ¹H-NMR (400 MHz, DMSO): δ/ppm =7.1 (s, 1H), 7.72 (dd, 1H), 8.33 (s, 1H),8.56 (s,1H), 8.65 (d, 1H).
b.p.: 140° C./10 mbar
b) 3-Chloro-2-[5-(trifluoromethyl)-1H-pyrazol-1yl]pyridine
The compound was isolated from a sample of the crude product by preparative chromatography and characterized by NMR spectroscopy.

¹³C-NMR (125 MHz, CDCl₃): δ/ppm=108.67 (d), 119.56 (q, ¹J (C,F)=269.2 Hz), 126.33 (d), 129.60 (s), 133.34 (q, ²J (C,F)=40.1 Hz), 139.65(d), 140.40 (d), 146.93 (d), 147.93 (d) ¹H-NMR (400 MHz, CDCl₃): δ/ppm=6.86 (s, 1H), 7.51 (dd, 1H), 7.82 (s, 1H), 7.96 (d,1H), 8.54 (d, 1H).

### m.p.: 40-41° C.

c) 2-(3-chloro-2-pyridyl)-3-(trifluoromethyl)-4H-pyrazol-3-ole
The compound was isolated from the reaction mixture before addition of the acid, by preparative column chromatography.
¹³C-NMR (125 MHz, CDCl₃): δ/ppm=45,76 (t), 92.64 (q, ²J (C,F)=32.8 Hz), 120.90 (d), 123.69 (s), 125.12 (q, ¹J (C,F)=285.0 Hz), 140.48(d), 141.42 (d), 143.43 (d), 153.89 (s) ¹H-NMR (500 MHz, CDCl₃): δ/ppm=3.14 (d, 1H), 3.39 (d, 1H), 7.05 (s, 1H), 7.12 (dd, 1H), 7.85 (d, 1H), 8.13 (d, 1H), 8.53 (s, broad OH) m.p.: 63° C.

### Example 2

21.4 g (3-Chloro-2-pyridyl)hydrazine (95.9 wt-%) were suspended in 210 g toluene. Then, at room temperature (25° C.), 4.2 g concentrated sulfuric acid (0.3 equivalents) were added. After that, 25.2 g ETFBO were added, and the mixture was heated under reflux for 1 hour. After cooling to 25° C., a small phase of reaction water was observed, which was removed. The organic phase contained (HPLC control) the isomers 3-chloro-2-[3-(trifluoromethyl)-1H-pyrazol-1yl]pyridine and 3-Chloro-2-[5-(trifluoromethyl)-1H-pyrazol-1yl]pyridine in a ratio of 63:1.
After washing of the organic phase with saturated NaHCO₃ solution and water, and removal of the solvent, 35.4 g of a red-brownish clear oil were obtained (quantitative HPLC: 93.7wt % of the desired isomer 3-chloro-2-[3-(trifluoromethyl)-1H-pyrazol-1yl]pyridine), yield 93.7%.

### Example 3

21.0 g (3-Chloro-2-pyridyl)hydrazine (97.8 wt-%) were suspended in 210 g toluene. Then, at room temperature (25° C.), 2.1 g concentrated sulfuric acid (0.15 equivalents) were added. After that, 25.2 g ETFBO were added, and the mixture was heated under reflux for 23 hours. After cooling to 25° C., a small phase of reaction water was observed, which was removed. The organic phase contained (HPLC control) the isomers 3-chloro-2-[3-(trifluoromethyl)-1H-pyrazol-1yl]pyridine and 3-Chloro-2-[5-(trifluoromethyl)-1H-pyrazol-1yl]pyridine in a ratio of 23:1.
After washing of the organic phase with saturated NaHCO₃ solution and water, and removal of the solvent, 34.9 g of a red-brownish clear oil were obtained (quantitative HPLC: 93.65wt % of the desired isomer 3-chloro-2-[3-(trifluoromethyl)-1H-pyrazol-1yl]pyridine), yield 92.3%.

### Example 4

21.0 g (3-Chloro-2-pyridyl)hydrazine (97.8 wt-%) were suspended in 210 g toluene. Then, at room temperature (25° C.), 4.2 g concentrated sulfuric acid (0.3 equivalents) were added. After that, the mixture was heated to reflux, and 25.2 g ETFBO were added over 2 hours. The mixture was heated under reflux for a total of 19 hours. After cooling to 25° C., a small phase of reaction water was observed, which was removed.
After washing of the organic phase with saturated NaHCO₃ solution and water, and removal of the solvent, 34.5 g of a red-brownish clear oil were obtained (quantitative HPLC: 93.6wt % of the desired isomer 3-chloro-2-[3-(trifluoromethyl)-1H-pyrazol-1yl]pyridine), yield 91.2%.

### Example 5

20.6 g (3-Chloro-2-pyridyl)hydrazine (100 wt-%) were suspended in 210 g toluene. Then, at room temperature (25° C.), 27.11 g concentrated hydrochloric acid (ca 2 equivalents) were added. After that, 25.2 g ETFBO were added, and after 15 minutes, the mixture was heated under reflux for 1 hour. After cooling to 25° C., a small phase of reaction water was observed, which was removed. After washing of the organic phase with saturated NaHCO₃ solution and water, and removal of the solvent, 34.3 g of a red-brownish clear oil were obtained (quantitative HPLC: 94.95 wt % of the desired isomer 3-chloro-2-[3-(trifluoromethyl)-1H-pyrazol-1yl]pyridine), yield 91.7%.

### Example 6

20.6 g (3-Chloro-2-pyridyl)hydrazine (100 wt-%) were suspended in 210 g toluene. Then, at room temperature (25° C.), 14.0 g concentrated sulfuric acid (ca 1 equivalent) were added. After that, 25.2 g ETFBO were added, and after 15 minutes, the mixture was heated under reflux for 3 hours. After cooling to 25° C., a small phase of reaction water was observed, which was diluted with 100 g water and removed. The organic phase contained (HPLC control) the isomers 3-chloro-2-[3-(trifluoromethyl)-1H-pyrazol-1yl]pyridine and 3-Chloro-2-[5-(trifluoromethyl)-1H-pyrazol-1yl]pyridine in a ratio of 40:1. After washing of the organic phase with saturated NaHCO₃ solution and water, and removal of the solvent, 33.0 g of a red-brownish clear oil were obtained (quantitative HPLC: 93.65 wt % of the desired isomer 3-chloro-2-[3-(trifluoromethyl)-1H-pyrazol-1yl]pyridine), yield 87.0%.

### Example 7

210 g toluene were mixed at room temperature (25° C.) with 13.8 g concentrated hydrochloric acid (ca 1 equivalent). Then, 25.2 g ETFBO were added. After stirring for 30 min, 20.6 g (3-Chloro-2-pyridyl) hydrazine (100 wt %) were added and heated under reflux. HPLC control of a sample taken during heating at 55° C.: proof of formation of 2-(3-chloro-2-pyridyl)-3-(trifluoromethyl)-4H-pyrazol-3-ole).

HPLC control of a sample taken after 1 hour under reflux: proof of formation of 3-chloro-2-[3- (trifluoromethyl)-1H-pyrazol-1yl]pyridine).

After cooling to 25° C., a small phase of reaction water was observed, which was removed. After washing of the organic phase with saturated NaHCO₃ solution and water, and removal of the solvent, 33.7 g of a red-brownish clear oil were obtained (quantitative HPLC: 96.1 wt % of the desired isomer 3-chloro-2-[3-(trifluoromethyl)-1H-pyrazol-1yl]pyridine), yield 91.2%.

### Example 8

154.5 g (3-Chloro-2-pyridyl)hydrazine (100 wt-%) were suspended in 1565 g toluene and heated up to 80° C. Then, 117 g concentrated hydrochloric acid (ca 1.1 equivalents) and 27 g water were added to obtain a biphasic liquid/liquid mixture. After that, 183 g ETFBO calc. 98.9 wt-% were dosed in during 30 min. During dosage the temperature increased to 87° C., which results in a slight reflux. After dosage the mixture is kept close below reflux at 85° C. for 1 h. After cooling to 25° C. the reaction water-phase was separated. The toluene solution was washed first with a mixture of 500 g water and 50 g NaOH (10 wt-%) solution and second with 750 g water. After washing the organic phase was concentrated at 50° C./ 1 mbar. 264,7 g of a clear orange oil were obtained (quantitative HPLC: 97 wt-% of the desired isomer 3-chloro-2-[3-(trifluoromethyl)-1H-pyrazol-1yl]pyridine), yield 96%.

### Example 9

150 g toluene were cooled to 10° C. Then 29 g trifluoracetyl chloride were introduced as gas. Then a mixture of 16.5 g ethylvinylether and 17.4 g pyridine were added over 30 min, which leads to a temperature increase to 15° C. After dosage the mixture was kept 2 h at 10° C. and 2 h at 25° C. 150 g water were added to dissolve the precipitated salts. After removal of the water-phase the toluene solution was dosed over 30 min to a hot (80° C.) mixture of 31,5 g (3-Chloro-2-pyridyl)hydrazine (100 wt-%), 22,8 g concentrated hydrochloric acid, 5.3 water and 179 g toluene. The resulting reaction mixture was kept 1 h at 85° C. After cooling to 25° C., the water-phase was removed. After washing of the organic phase with 150 g saturated NaHCO₃ solution and 150 g water, and removal of the solvent, 35.3 g of a red-brownish clear oil were obtained (quantitative HPLC: 88 wt % of the desired isomer 3-chloro-2-[3-(trifluoromethyl)-1H-pyrazol-1yl]pyridine), yield 57% based on trifluoracetyl chloride.

A detailed description, how the compounds of formula (I) can be converted to the compounds of formula (I-A), (I-B), and necessary intermediates, can be found in WO2013/076092. Following the procedures given there, and analogous methods, the following compounds of formula (I-B-1) can be synthesized, which are are compounds of the formula (I-B) with k=0 and R³═H.

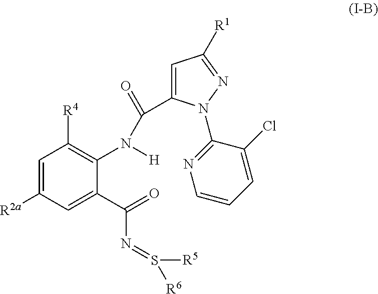

| Ex. I-B | R⁵ | R⁶ | R⁴ | R^{2a} | R¹ |
|---|---|---|---|---|---|
| 1 | C₂H₅ | C₂H₅ | Cl | Cl | CF₃ |
| 2 | CH(CH₃)₂ | CH(CH₃)₂ | Cl | Cl | CF₃ |
| 3 | CH₃ | CH₃ | Cl | Cl | CF₃ |

| | | | | |
|---|---|---|---|---|
| 4 | CH₂CH₂CH₂CH₂ | Cl | Cl | CF₃ |

| | | | | | |
|---|---|---|---|---|---|
| 5 | CH₃ | CH₃ | CH₃ | Cl | CF₃ |
| 6 | C₂H₅ | C₂H₅ | CH₃ | Cl | CF₃ |
| 7 | CH(CH₃)₂ | CH(CH₃)₂ | CH₃ | Cl | CF₃ |

| | | | | |
|---|---|---|---|---|
| 8 | CH₂CH₂CH₂CH₂ | CH₃ | Cl | CF₃ |

| | | | | | |
|---|---|---|---|---|---|
| 9 | C₂H₅ | C₂H₅ | Br | Cl | CF₃ |
| 10 | CH(CH₃)₂ | CH(CH₃)₂ | Br | Cl | CF₃ |
| 11 | C₂H₅ | C₂H₅ | Br | Br | CF₃ |
| 12 | CH(CH₃)₂ | CH(CH₃)₂ | Br | Br | CF₃ |
| 13 | C₂H₅ | C₂H₅ | CF₃ | Cl | CF₃ |
| 14 | CH(CH₃)₂ | CH(CH₃)₂ | CF₃ | Cl | CF₃ |
| 15 | C₂H₅ | C₂H₅ | CF₃ | Br | CF₃ |
| 16 | CH(CH₃)₂ | CH(CH₃)₂ | CF₃ | Br | CF₃ |
| 17 | C₂H₅ | C₂H₅ | Br | CF₃ | CF₃ |
| 18 | CH(CH₃)₂ | CH(CH₃)₂ | Br | CF₃ | CF₃ |
| 19 | C₂H₅ | C₂H₅ | Cl | CF₃ | CF₃ |
| 20 | CH(CH₃)₂ | CH(CH₃)₂ | Cl | CF₃ | CF₃ |
| 21 | C₂H₅ | C₂H₅ | Cl | CN | CF₃ |
| 22 | CH(CH₃)₂ | CH(CH₃)₂ | Cl | CN | CF₃ |
| 23 | C₂H₅ | C₂H₅ | CH₃ | CN | CF₃ |
| 24 | CH(CH₃)₂ | CH(CH₃)₂ | CH₃ | CN | CF₃ |

| | | | | |
|---|---|---|---|---|
| 25 | CH₂CH₂CH₂CH₂ | CH₃ | Cl | Br |

| | | | | | |
|---|---|---|---|---|---|
| 26 | CH₃ | CH₃ | CH₃ | Cl | Br |
| 27 | C₂H₅ | C₂H₅ | CH₃ | Cl | Br |
| 28 | CH(CH₃)₂ | CH(CH₃)₂ | CH₃ | Cl | Br |

| | | | | |
|---|---|---|---|---|
| 29 | CH₂CH₂CH₂CH₂ | Cl | Cl | Br |

| | | | | | |
|---|---|---|---|---|---|
| 30 | CH₃ | CH₃ | Cl | Cl | Br |
| 31 | C₂H₅ | C₂H₅ | Cl | Cl | Br |
| 32 | CH(CH₃)₂ | CH(CH₃)₂ | Cl | Cl | Br |

| | | | | |
|---|---|---|---|---|
| 33 | CH₂CH₂CH₂CH₂ | CH₃ | Cl | CHF₂ |

| | | | | | |
|---|---|---|---|---|---|
| 34 | CH₃ | CH₃ | CH₃ | Cl | CHF₂ |
| 35 | C₂H₅ | C₂H₅ | CH₃ | Cl | CHF₂ |
| 36 | CH(CH₃)₂ | CH(CH₃)₂ | CH₃ | Cl | CHF₂ |

| | | | | |
|---|---|---|---|---|
| 37 | CH₂CH₂CH₂CH₂ | Cl | Cl | CHF₂ |

| | | | | | |
|---|---|---|---|---|---|
| 38 | CH₃ | CH₃ | Cl | Cl | CHF₂ |
| 39 | C₂H₅ | C₂H₅ | Cl | Cl | CHF₂ |
| 40 | CH(CH₃)₂ | CH(CH₃)₂ | Cl | Cl | CHF₂ |
| 41 | C₂H₅ | C₂H₅ | Br | Cl | CHF₂ |
| 42 | CH(CH₃)₂ | CH(CH₃)₂ | Br | Cl | CHF₂ |
| 43 | C₂H₅ | C₂H₅ | Br | Br | CHF₂ |
| 44 | CH(CH₃)₂ | CH(CH₃)₂ | Br | Br | CHF₂ |
| 45 | C₂H₅ | C₂H₅ | CF₃ | Cl | CHF₂ |
| 46 | CH(CH₃)₂ | CH(CH₃)₂ | CF₃ | Cl | CHF₂ |
| 47 | C₂H₅ | C₂H₅ | CF₃ | Br | CHF₂ |
| 48 | CH(CH₃)₂ | CH(CH₃)₂ | CF₃ | Br | CHF₂ |
| 49 | C₂H₅ | C₂H₅ | Br | CF₃ | CHF₂ |
| 50 | CH(CH₃)₂ | CH(CH₃)₂ | Br | CF₃ | CHF₂ |
| 51 | C₂H₅ | C₂H₅ | Cl | CF₃ | CHF₂ |
| 52 | CH(CH₃)₂ | CH(CH₃)₂ | Cl | CF₃ | CHF₂ |
| 53 | C₂H₅ | C₂H₅ | Cl | CN | CHF₂ |
| 54 | CH(CH₃)₂ | CH(CH₃)₂ | Cl | CN | CHF₂ |
| 55 | C₂H₅ | C₂H₅ | CH₃ | CN | CHF₂ |
| 56 | CH(CH₃)₂ | CH(CH₃)₂ | CH₃ | CN | CHF₂ |

For the details of the insecticidal properties of the compounds of formula (I-B), see e,g, WO2007/006670, WO2013/024009, and WO2013/024010.

1-16. (canceled)

17. A process for preparing a pyridylpyrazole compound of the formula (I) 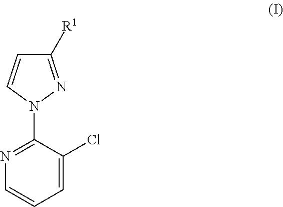
in which R¹ is selected from CF₃ and CHF₂;
comprising the step of reacting a compound of the formula (II) 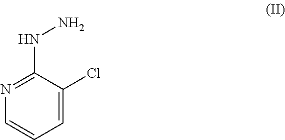
with a compound of formula (III) 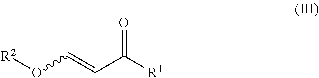
wherein R¹ is as defined above;
and R² is selected from C₁-C₆-alkyl, C₂-C₆-cycloalkyl, aralkyl and aryl;
in the presence of an acid.

18. The process according to claim 17, wherein the process goes via the intermediate of formula (IV): 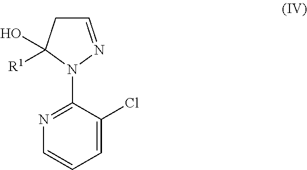
in which R¹ is selected from CF₃ and CHF₂.

19. The process according to claim 17, in which the acid is selected from hydrochloric acid HCl, sulfuric acid H₂SO₄ and phosphoric acid H₃PO₄.

20. The process according to claim 17, in which the acid is an aqueous acid.

21. The process according to claim 17, in which the reaction is carried out in a solvent selected from toluene, ethylbenzene, o-xylene, m-xylene, p-xylene, chlorobenzene, or a mixture thereof.

22. The process according to claim 17, in which the reaction is carried out at a temperature between 60 and 120° C.

23. The process according to claim 17, in which R¹ is CF₃.

24. The process according to claim 17, wherein the compound of the formula (II) 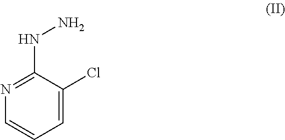
is prepared in step (i) by reacting dichloropyridine compound (VI) 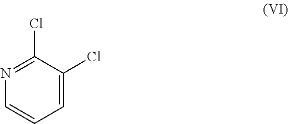
with hydrazine,
followed by the step (ii).

25. The process according to claim 17, wherein the compound of the formula (III) 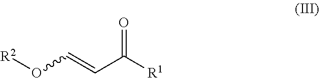
is prepared by reacting the vinyl ether (IIIa) 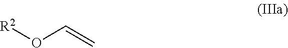
with a reagent selected from trifluoro-/difluoroacetyl chloride, trifluoro-/difluoroacetyl bromide, or trifluoro-/difluoroacetyl anhydride
and is provided for step (ii) of claim 17 as a crude product, optionally together with the primary conversion products of formula (IIIb) 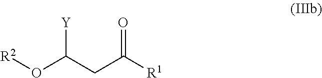
in which Y is chloro or bromo, and R¹ is as defined in any of the preceding claims,
followed by the step (ii).

26. A process for preparing a compound of formula (I-A) 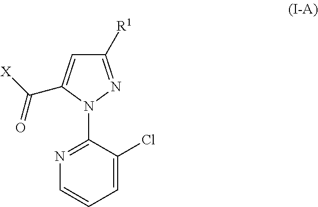
wherein
R^{t} is selected from CF₃ and CHF₂;
X is selected from halogenOH, O—Mg—Cl, O—Mg—Br, imidazole, —O—CO—R^{x}, —O—CO—OR^{x}, —OSO₂R^{x}, —SR^{y}, in which
R^{x} is independently selected from C₁-C₆-alkyl, trifluoromethyl and phenyl which is optionally substituted with C₁-C₆-alkyl or halogen, and
R^{y} is independently selected from C₁-C₆-alkyl and phenyl which is optionally substituted with C₁-C₆-alkyl or halogen;
the process comprising:
a) providing the compound of the formula (I) by a process according to claim 17, 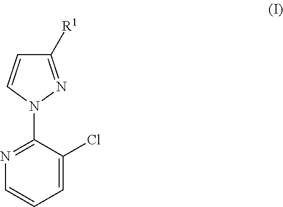
b) reacting the compound of formula (I) in a step (iii) to the corresponding carbonyl compound of formula (I-A).

27. The process according to claim 26, comprising the steps of iii-a) deprotonating a compound of the formula (I) 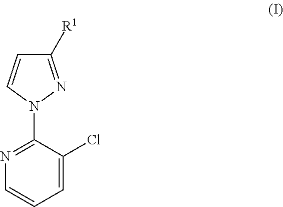
with a magnesium-organic base having a carbon bound magnesium, or with a magnesium amide having a nitrogen bound magnesium which is derived from a secondary amine, in the presence of a lithium halide, where the base is used in an amount sufficient to achieve at least 80% deprotonation of the compound of formula (I); and
iii-b) subjecting the product obtained in step (iii-a) to a carboxylation by reacting it with a reagent selected from phosgene and carbon dioxide, to obtain a compound of formula (I-A) as defined above.

28. A process for preparing an anthranilamide compound of formula (I-B): 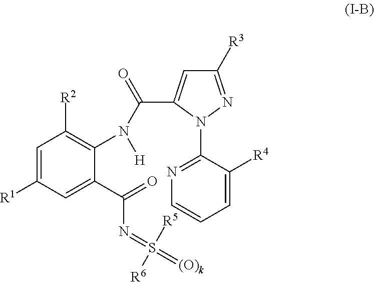
wherein
R¹ is selected from the group consisting of H, F, Cl, Br and CN;
R² is selected from the group consisting of F, Cl, Br, I, CH₃;
R³ is selected from the group consisting of Br, Cl, CHF₂, CF₃ and OCH₂F;
R⁴ is Cl or CF₃;
R⁵, R⁶ are selected independently of one another from the group consisting of hydrogen, C₁-C₄-alkyl, C₃-C₈-cycloalkyl, or
R⁵ and R⁶ together represent a C₂-C₇-alkylene, C₂-C₇-alkenylene or
C₆-C₉-alkynylene chain forming together with the sulfur atom to which they are attached a 3-, 4-, 5-, 6-, 7-, 8-, 9- or 10-membered saturated, partially unsaturated or fully unsaturated ring,
k is 0 or 1;
or a stereoisomer, salt, tautomer or N-oxide, or a polymorphic crystalline form, a co- crystal or a solvate of a compound or a stereoisomer, salt, tautomer or N-oxide thereof;
the process comprising
a) providing a compound of the formula (I) by a process according to claim 17,
b) converting the compound of formula (I) to a compound of formula (I-B), optionally via the corresponding carbonyl compound of formula (I-A) as defined in claim 10.

29. The process according to claim 28, wherein the process comprises
a) providing a compound of the formula (I) by a process according to claim 17,
b) reacting the compound of formula (I) in a step (iii) to the corresponding carbonyl compound of formula (I-A), 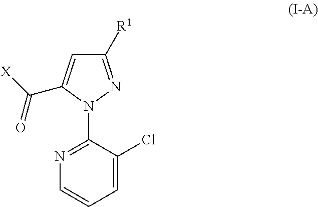
wherein
R¹ is selected from the group consisting of H, F, Cl, Br and CN;
X is selected from halogen, OH, O—Mg—Cl, O—Mg—Br, imidazole, —O—CO—R^{x}, —O—CO—OR^{x}, —OSO₂R^{x}, —SR'';
c) converting the compound of formula (I-A) in a step (iv) to a compound of formula (I-B): 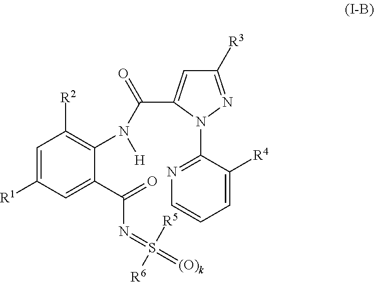
R² is selected from the group consisting of F, Cl, Br, I, CH₃;
R³ is selected from the group consisting of Br, Cl, CHF₂, CF₃ and OCH₂F;
R⁴ is Cl or CF₃;
R⁵, R⁶ are selected independently of one another from the group consisting of hydrogen, C₁-C₄-alkyl, C₃-C₈-cycloalkyl, or
R⁵ and R⁶ together represent a C₂-C₇-alkylene, C₂-C₇-alkenylene or
C₆-C₉-alkynylene chain forming together with the sulfur atom to which they are attached a 3-, 4-, 5-, 6-, 7-, 8-, 9- or 10-membered saturated, partially unsaturated or fully unsaturated ring,
k is 0 or 1.

30. The process according to claim 28, wherein the compound of formula (I-B) is selected from the the group consisting of the following compounds I-11, I-16, I-21, I-26, I-31:

| | R¹ | R² | R³ | R⁵ | R⁶ | k |
|---|---|---|---|---|---|---|
| I-11 | Cl | CH₃ | CF₃ | C₂H₅ | C₂H₅ | 0 |
| I-16 | Cl | Cl | CF₃ | C₂H₅ | C₂H₅ | 0 |
| I-21 | Cl | CH₃ | CF₃ | CH(CH₃)₂ | CH(CH₃)₂ | 0 |
| I-26 | Cl | Cl | CF₃ | CH(CH₃)₂ | CH(CH₃)₂ | 0 |
| I-31 | Br | Br | CF₃ | C₂H₅ | C₂H₅ | 0 |

31. The process according to claim 29, wherein step (iv) in c) comprises iv) reacting the compound of the formula (I-A) with a compound of the formula (V) 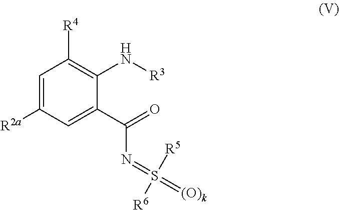
in which
R^{2a} is selected from the group consisting of hydrogen, halogen, halomethyl and cyano;
R³ is selected from hydrogen, C₁-C₆ alkyl;
R⁴ is selected from the group consisting of halogen, methyl and halomethyl;
R⁵, R⁶ are selected independently of one another from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present;
k is 0 or 1;
in the presence of a base, to obtain a compound of the formula (I-B; 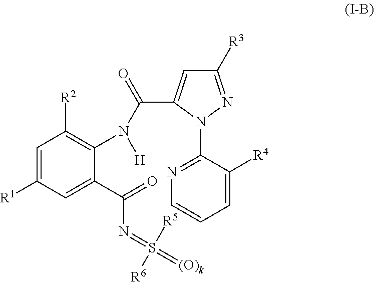
R¹ is selected from the group consisting of hydrogen, halogen, halomethyl and cyano;
R² is selected from the group consisting of halogen, methyl and halomethyl.

32. A compound of formula (IV) 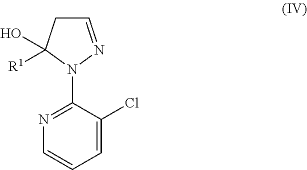
in which R¹ is selected from CF₃ and CHF₂.
